(19) United States
(12) Patent Application Publication
(10) Pub. No.: US 2025/0144361 A1
(43) Pub. Date: May 8, 2025

(54) PREPARING A PATIENT SUFFERING FROM BURNOUT TO RETURN TO WORK WITH A THERAPEUTIC PROCESS BY APPLYING EXPOSURE THERAPY USING VIRTUAL REALITY

(71) Applicant: Melimpus, Bruxelles (BE)

(72) Inventors: Stéphanie DELROISSE, Lillois (BE); Gaetan VAN WIJCK, Bruxelles (BE); Arnaud BOSTEELS, Tervuren (BE)

(73) Assignee: Melimpus, Bruxelles (BE)

(21) Appl. No.: 18/503,588

(22) Filed: Nov. 7, 2023

(51) Int. Cl. A61M 21/02; G06F 3/01

(52) U.S. Cl. CPC A61M 21/02; G06F 3/011

(57) ABSTRACT

Methods and systems for preparing a patient suffering from burnout to return to work with a therapeutic process by applying exposure therapy, including: applying a first scenario protocol, including: presents a first virtual reality scenario representing a first return-to-office scenario, including one or more burnout related triggers at a first level; applying a second scenario protocol, including: presents a second virtual reality scenario representing a second return-to-office scenario, including one or more burnout related triggers at a second level; wherein the burnout related triggers at the second level in the second return-to-office scenario are at a higher stress level in comparison to the burnout related triggers at the first level in the first return-to-office scenario.

### BACKGROUND OF THE INVENTION

### Field of the Invention

The present invention relates to the field of treating patients suffering from burnout. The invention relates in particular to a therapeutic process or a care pathway in which the patient is exposed to exposure therapy, using a virtual reality system. The therapeutic process or care pathway can e.g. be part of a therapy. The present invention more generally relates to the field of assisting people to return to work after an absence. The absence may e.g. be burnout-related, stress-related, or related to a common mental disorder.

### Description of the Related Art

Over the last decades, more and more people are diagnosed with burnouts. Different definitions are used to describe burnout, but generally burnout relates to a state of emotional, physical, and mental exhaustion. The causes for the burnout can vary greatly, but a common type of burnout is occupational burnout, where one of the contributing aspects is the work environment (e.g. the office) of the patient. The work environment can have many triggers that induce stress at the patient. As currently for example defined by the World Health Organization (WHO), occupational burnout is a phenomenon resulting from chronic work-related stress, with symptoms characterized by “feelings of energy depletion or exhaustion; increased mental distance from one's job, or feelings of negativism or cynicism related to one's job; and reduced professional efficacy”.

Known treatments of burnout include rest, stress-reducing activities, and therapy. The patient will usually stay home from work until the symptoms are considered to have reduced sufficiently to return to work. However, it is very difficult to determine when the patient is ready to return to work. Moreover, shortly after re-entering the work environment, the patient will be exposed again to many of the triggers that contributed to the burnout in the first place. If the patient returns to work too quickly and/or is exposed to too many triggers to quickly after returning, a relapse into the burnout may occur.

### BRIEF SUMMARY OF THE INVENTION

It is an object of the invention to overcome the disadvantages of the prior art, or at least provide an alternative to the prior art. It is in particular an object of the invention to provide a solution that allows to expose the patient to triggers in a controlled way.

One or more objects are achieved with a method for preparing a patient suffering from burnout to return to work with a therapeutic process by applying exposure therapy, including:
applying a first scenario protocol, including:
controlling, using a practitioner control system, when a virtual reality system including virtual reality glasses presents a first virtual reality scenario to a patient, wherein:
the first virtual reality scenario represents a first return-to-office scenario, wherein the first return-to-office scenario includes one or more burnout related triggers at a first level;
presenting the first virtual reality scenario to the patient using the virtual reality glasses arranged on the patient, wherein the patient virtually experiences the first virtual reality scenario in first person;
while the first virtual reality scenario is being presented to the patient by the virtual reality system, visually presenting the first virtual reality scenario to a practitioner using a screen;
applying a second scenario protocol, including:
controlling, using the practitioner control system, when the virtual reality system including virtual reality glasses presents a second virtual reality scenario to the patient, wherein:
the second virtual reality scenario represents a second return-to-office scenario, wherein the second return-to-office scenario includes one or more burnout related triggers at a second level;
presenting the second virtual reality scenario to the patient using the virtual reality glasses arranged on the patient, wherein the patient virtually experiences the second virtual reality scenario in first person;
while the second virtual reality scenario is being presented to the patient by the virtual reality system, visually presenting the second virtual reality scenario to the practitioner using the screen;
wherein
the burnout related triggers at the second level in the second return-to-office scenario are at a higher stress level in comparison to the burnout related triggers at the first level in the first return-to-office scenario.

One or more objects are achieved with a method for preparing a patient suffering from burnout to return to work with a therapeutic process by applying exposure therapy, including:
controlling a practitioner control system to:
allow a practitioner to control a virtual reality system including virtual reality glasses when to present a first virtual reality scenario and when to present a second virtual reality scenario;
controlling a virtual reality system to, in function of the control provided by the practitioner control system:
present the first virtual reality scenario to the patient using the virtual reality glasses arranged on the patient, wherein
the patient virtually experiences the first virtual reality scenario in first person;
the first virtual reality scenario represents a first return-to-office scenario, wherein the first return-to-office scenario includes one or more burnout related triggers at a first level;
present the second virtual reality scenario to the patient using the virtual reality glasses arranged on the patient, wherein
the patient virtually experiences the second virtual reality scenario in first person;
the second virtual reality scenario represents a second return-to-office scenario, wherein the second return-to-office scenario includes one or more burnout related triggers at a second level;
control the practitioner control system to:
while the first virtual reality scenario is being presented to the patient by the virtual reality system, visually present the first virtual reality scenario to a practitioner using a screen;
while the second virtual reality scenario is being presented to the patient by the virtual reality system, visually present the second virtual reality scenario to the practitioner using the screen;
wherein
the burnout related triggers at the second level in the second return-to-office scenario are at a higher stress level in comparison to the burnout related triggers at the first level in the first return-to-office scenario.

One or more objects of the invention are achieved with a system for preparing a patient suffering from burnout to return to work with a therapeutic process by applying exposure therapy, including:
a practitioner control system configured to:
allow a practitioner to control a virtual reality system including virtual reality glasses when to present a first virtual reality scenario and when to present a second virtual reality scenario;
a virtual reality system configured to, in function of the control provided by the practitioner control system:
present the first virtual reality scenario to the patient using the virtual reality glasses arranged on the patient, wherein:
the patient virtually experiences the first virtual reality scenario in first person;
the first virtual reality scenario represents a first return-to-office scenario, wherein the first return-to-office scenario includes one or more burnout related triggers at a first level;
present the second virtual reality scenario to the patient using the virtual reality glasses arranged on the patient,
the patient virtually experiences the second virtual reality scenario in first person;
the second virtual reality scenario represents a second return-to-office scenario, wherein the second return-to-office scenario includes one or more burnout related triggers at a second level
wherein the practitioner control system is further configured to:
while the first virtual reality scenario is being presented to the patient by the virtual reality system, visually present the first virtual reality scenario to a practitioner using a screen;
while the second virtual reality scenario is being presented to the patient by the virtual reality system, visually present the second virtual reality scenario to the practitioner using the screen;
wherein
the burnout related triggers at the second level in the second return-to-office scenario are at a higher stress level in comparison to the burnout related triggers at the first level in the first return-to-office scenario.

The invention can thus relate to methods and/or systems. Although the methods as described herein can be performed with the systems as described herein; neither the systems, nor the methods are limited thereto. Features explained herein with reference to the systems have the same meaning with respect to the methods unless explicitly defined otherwise, and vice versa. Features explained with reference to the systems can be applied mutatis mutandis to the method to achieve the similar advantages, and vice versa.

One or more objects of the invention can further achieved with non-transitory computer readable instructions, configured to cause a system for preparing a patient suffering from burnout to return to work with a therapeutic process by applying exposure therapy (e.g. according to any of the embodiments described herein) to perform a method for preparing a patient suffering from burnout to return to work with a therapeutic process by applying exposure therapy (e.g. according to any of the embodiments described herein).

Embodiments of the methods and system described herein thus relate to preparing a patient suffering from burnout to return to work. The patient is a human, e.g. an adult. The patient may be diagnosed with a burnout, e.g. an occupational burnout, e.g. resulting from chronic work-related stress. An occupational burnout can e.g. be defined as the definition by the World Health Organization (WHO): occupational burnout is a phenomenon resulting from chronic work-related stress, with symptoms characterized by “feelings of energy depletion or exhaustion; increased mental distance from one's job, or feelings of negativism or cynicism related to one's job; and reduced professional efficacy”. A burnout is not a depression or a phobia.

The work of the patient may in particular be a job in an office environment, e.g. a desk job. The desk job can e.g. entail that at least a part of the job is performed behind a desk and/or using a computer. However, it may be possible that a part of the desk job entails different tasks, e.g. visiting prospects/clients and/or constructions sites. The office can e.g. be an open office. The office can e.g. be a home office, e.g. for part of the job. The patient can be prepared for returning to the same job or a different job, e.g. a different role at the same company or a role at a different company.

Optionally, a therapeutic process and/or a care pathway is provided for preparing the patient. The therapeutic process and/or care pathway can e.g. be part of a therapy, wherein the therapy e.g. includes applying various scenario protocols and/or showing the patient various virtual reality scenarios. The care pathway is e.g. a mutually agreed framework (between patient and practitioner) for the decision-making and organization of healthcare processes for one or more patients during a given period. The care pathway may thus be broader than the virtual reality scenarios, and also include the follow-up by the practitioner during and after the scenario protocols. However, the invention may also relate to a non-therapeutic process in some embodiments, e.g. for training a patient.

The therapeutic process includes applying exposure therapy. Exposure therapy entails that the patient is exposed to a trigger (or stimulus), wherein in this case the trigger is a burnout related trigger. The patient is exposed to the trigger in a safe environment, as is explained in more detail below. This allows the patient to manage and reprogram his reaction to the triggers. In this particular case, this can e.g. result in the patient being less stressed by the triggers, such that the likelihood of a relapse of the burnout can be reduced.

A first and optionally a second virtual reality scenario can be presented to the patient. A virtual reality system is configured to present the respective virtual reality scenarios to the patient, e.g. visually and optionally also auditory. The virtual reality scenario is a 3-dimensional (3D), immersive experience to which the patient is exposed. The virtual reality system can e.g. include virtual reality glasses, which can e.g. be part of headset or a head mounted display (HMD). Optionally, the virtual reality system further includes a sound emitter, e.g. as part of the same headset of HMD.

The virtual reality system is configured to apply virtual reality. Virtual reality can e.g. be defined as the computer-generated simulation of a three-dimensional image or environment that can be interacted with in a seemingly real or physical way by a person using special electronic equipment, such as a helmet with a screen inside. Virtual reality can e.g. be defined as a simulated experience that employs pose tracking and 3D near-eye displays to give the user an immersive feel of a virtual world. Virtual reality can e.g. be defined as a simulated 3D environment that enables users to explore and interact with a virtual surrounding in a way that approximates reality, as it is perceived through the users' senses. The environment or images within the virtual reality can e.g. cover 360 degrees.

The virtual reality system is configured to present respective virtual reality scenarios to the patient in first person. The patient thus experiences the virtual reality scenarios as if he is an active participant in the scenario. The first scenario represents a first return-to-office scenario, and the second scenario represents a second return-to-office scenario. A return-to-office scenario is a scenario that could occur when the patient returns to work, e.g. on the first day or the first few days. Examples of such scenarios are elaborated on further below. The return-to-office scenarios can e.g. occur in a work environment, e.g. in an office environment or in a home office environment. Although the work environment will usually not be an exact (virtual) replica of the work environment of the patient, it may include enough resemblance for the patient to make the link to his own work environment.

Each return-to-office scenario preferably includes one or more burnout related triggers. A burnout related trigger is something that would trigger stress at the patient, which could contribute to or reinforce the burnout. Examples of such triggers are elaborated on further below. The patient can thus be exposed to triggers similar to those that caused the burnout in real life, while remaining in the safe and controlled environment of the practitioner. This allows the patient to reprogram his reaction to such triggers.

Different triggers can cause stress at different levels. A level can as such be contributed to the triggers. The first return-to-office scenario includes one or more burnout related triggers at a first level; and the second return-to-office scenario includes one or more burnout related triggers at a second level. The burnout related triggers at the second level in the second return-to-office scenario are at a higher stress level in comparison to the burnout related triggers at the first level in the first return-to-office scenario. Thus, depending on which virtual reality scenario is shown to the patient, the level of possibly induced stressed is different. This allows to adapt the therapeutic process to the needs of the patient.

Although in practice it may be difficult to objectively assign a stress level to a trigger in absolute terms, it is pointed out that in the context of this description, the stress levels are considered in relative terms, i.e. one being higher than the other. As will be clear from the examples of triggers given further below, the different return-to-office scenarios can include different triggers, of which it would generally be understood by the population which triggers causes a higher stress level. If needed, different virtual reality scenarios and/or triggers can be ranked in relative order of stress level by performing a survey. For example, a sufficiently large test group (e.g. at least 15 people, or at least 30, 50, 100, 1000) can be asked to order the virtual reality scenarios and/or triggers. When at least 70% (e.g. at least 80%, or at least 90%) of the people rank a virtual reality scenario and/or trigger to trigger a higher stress level than another virtual reality scenario and/or trigger, it will be considered that the relative stress levels are accordingly. The people can e.g. be exposed to the virtual reality scenarios and/or triggers by use of a virtual reality system, or it can be explained to them (e.g. in written or oral form).

A practitioner control system is configured to control the virtual reality system. The practitioner control system is configured to be controlled by a practitioner. The practitioner is the person treating and/or preparing the patient to return to work. The practitioner can e.g. be a therapist, a psychologist, or a trained professional. The practitioner can thus control the virtual reality system by using the practitioner control system.

The practitioner control system at least allows the practitioner to control when which virtual reality scenario is presented to the patient. The practitioner can as such control when the patient is exposed to which burnout related triggers, in particular which level of burnout related the triggers. The practitioner can as such adapt the therapeutic process of the patient, based on suitable factors. Such factors can e.g. include the progress of the patient, which virtual reality scenario has been shown previously, the response of the patient to previous virtual reality scenario(s), how long the patient has been following therapy, etc. The practitioner control system can e.g. be configured to allow the practitioner to select a virtual reality scenario, and optionally select options that affect the virtual reality scenario. The options can e.g. relate to the environment of the virtual reality scenario and/or whether or not certain scenes are incorporated in the virtual reality scenario. The practitioner control system can e.g. be configured to allow the practitioner to start pause, and/or stop a virtual reality scenario.

Optionally, the practitioner control system is configured to visually present the first or second virtual reality scenario to the practitioner, while the first or second virtual reality scenario, respectively, is presented to the patient by the virtual reality system. The respective virtual reality scenario can e.g. be presented to a practitioner by a screen. In some embodiments, the screen is a virtual reality screen. In some embodiments, the screen is not a virtual reality screen. The screen can e.g. be a screen of a computer, laptop, tablet, smartphone, television. Optionally, the practitioner control system includes the screen. Thus, the practitioner can follow along while the patient is experiencing the virtual reality scenario in virtual reality. This allows the practitioner to easier asses the reaction of the patient to the burnout related triggers.

The methods and system of the invention can thus include showing different virtual reality scenarios to the patient. Each scenario has burnout related triggers at different levels. The practitioner can control when which scenario is shown to the patient, and can follow along on the screen. The practitioner can as such control the therapeutic process, and subject the patient to the exposure therapy. The levels of the burnout related triggers can furthermore be adapted to the recovery level of the patient, until he is ready to return to work in real life. In the optional embodiments including the presenting of the virtual scenario to the practitioner, the practitioner can furthermore follow along and better monitor the reaction of the patient to the burnout related triggers.

In embodiments, multiple scenario protocols can be applied, e.g. by the practitioner. A respective scenario protocol can e.g. include controlling when the virtual reality scenario presented to the patient, presenting it to the patient, and while it is being presented to the patient also presenting it to the practitioner (e.g. via the screen). Optionally, a scenario protocol includes additional actions, e.g. subjecting the patient questions and/or an intellectual exercise. The practitioner can perform the scenario protocol at a first moment in time, and the second scenario protocol at a second moment in time. The second moment in time can e.g. after the first moment in time. The second moment in time can e.g. be on the same day as the first moment in time. The second moment can e.g. on a later day than the first moment in time.

In embodiments, the methods and/or systems may include and/or be configured to: present of a third virtual reality scenario representing a third return-to-office scenario including one or more burnout related triggers at a third level, wherein the third level is at a higher stress level in comparison to the burnout related triggers in the second return-to-office scenario. Optionally, the method and/or system may include applying a third scenario protocol including presenting the third virtual reality scenario. For example, the third scenario protocol may include: controlling, using the practitioner control system, when the virtual reality system including virtual reality glasses presents the third virtual reality scenario to the patient, wherein the third virtual reality scenario represents a third return-to-office scenario, presenting the third virtual reality scenario to the patient using the virtual reality glasses arranged on the patient, wherein the patient virtually experiences the third virtual reality scenario in first person; while the third virtual reality scenario is being presented to the patient by the virtual reality system, visually presenting the third virtual reality scenario to the practitioner using the screen. Optionally, the third virtual reality scenario is presented to the patient at a third moment in time, which may e.g. be after the second moment in time.

In embodiments, the methods and/or systems may include and/or be configured to: present of a fourth virtual reality scenario representing a fourth return-to-office scenario including one or more burnout related triggers at a fourth level, wherein the fourth level is at a higher stress level in comparison to the burnout related triggers in the third return-to-office scenario. Optionally, the method and/or system may include applying a fourth scenario protocol including presenting the fourth virtual reality scenario. For example, the fourth scenario protocol may include: controlling, using the practitioner control system, when the virtual reality system including virtual reality glasses presents the fourth virtual reality scenario to the patient, wherein the fourth virtual reality scenario represents a fourth return-to-office scenario, presenting the fourth virtual reality scenario to the patient using the virtual reality glasses arranged on the patient, wherein the patient virtually experiences the fourth virtual reality scenario in first person; while the fourth virtual reality scenario is being presented to the patient by the virtual reality system, visually presenting the fourth virtual reality scenario to the practitioner using the screen. Optionally, the fourth virtual reality scenario is presented to the patient at a fourth moment in time, which may e.g. be after the third moment in time.

In embodiments, the method and/or system may include and/or be configured to: present of a fifth virtual reality scenario representing a fifth return-to-office scenario including one or more burnout related triggers at a fifth level, wherein the fifth level is at a higher stress level in comparison to the burnout related triggers in the fourth return-to-office scenario. Optionally, the method and/or system may include applying a fifth scenario protocol including presenting the fifth virtual reality scenario. For example, the fifth scenario protocol may include: controlling, using the practitioner control system, when the virtual reality system including virtual reality glasses presents the fifth virtual reality scenario to the patient, wherein the fifth virtual reality scenario represents a fifth return-to-office scenario, presenting the fifth virtual reality scenario to the patient using the virtual reality glasses arranged on the patient, wherein the patient virtually experiences the fifth virtual reality scenario in first person; while the fifth virtual reality scenario is being presented to the patient by the virtual reality system, visually presenting the fifth virtual reality scenario to the practitioner using the screen. Optionally, the fifth virtual reality scenario is presented to the patient at a fifth moment in time, which may e.g. be after the third moment in time.

The invention can thus in embodiments include three, four, or five virtual scenarios and/or scenario protocols having burnout related triggers at different stress levels. This can e.g. allow to gradually increase the stress level while the patient improves, and/or to provide more flexibility in defining the therapeutic process. It will be understood that more than five virtual scenarios and/or scenario protocols can be provided in embodiments similar as those described above, mutatis mutandis.

In embodiments of the methods and/or systems, the first scenario protocol is applied at a point in time before the second scenario protocol is applied. Since the triggers in the second virtual reality scenario are at a higher level than in the first virtual reality scenario, this implies that the level of the triggers is increased. When more than two virtual reality scenarios are presented to the patient, the level of triggers may be further increased over time. For example, the third scenario protocol is applied at a point in time after the second scenario protocol is applied. For example, the fourth scenario protocol is applied at a point in time after the third scenario protocol is applied. For example, the fifth scenario protocol is applied at a point in time after the fourth scenario protocol is applied. These embodiments may be advantageous because the patient is subjected to relatively low stress levels at first. As the therapeutic process progresses, the patient may gradually improve and the stress levels are increased, until the patient is ready for a real-life experience and thus return to work.

In embodiments, the virtual reality system is able to present various different virtual reality scenarios to the patient. Each virtual reality scenario can e.g. include one or more scenes. Optionally, the practitioner control system is configured to allow the practitioner to select various parameters of the virtual reality scenario.

For example, the various parameters can include the environment in which the virtual reality scenario occurs. For example, the possible environments can include one or more of: an open space office, a meeting room, a home office. The open space office can e.g. be a room with a plurality (e.g. between 4-10) of desks (e.g. having at least a computer (screen) on it) for a plurality of colleagues. Optionally, the patient is seated at a desk in the back so that the other desks and colleagues are visible. Optionally the open office space has at least one window, which can e.g. serve as reflective surface in which the patient can see a reflection. The meeting room can e.g. have a large table with a plurality (e.g. 8-16) of chairs around the table. Optionally the meeting room has glass walls and/or a window, which can e.g. serve as reflective surface in which the patient can see a reflection. The meeting room can e.g. have one or more motivational posters on the walls, wherein the motivational posters depict principles that could be stress-triggers. The home office can e.g. be at a dining table or kitchen table in a house, wherein a laptop or computer is arranged on the the table. Optionally the home office has at least one window, which can e.g. serve as reflective surface in which the patient can see a reflection.

For example, the various parameters can include an attitude of one or more colleagues (avatars) in the virtual reality scenario. For example, one or more of the colleagues can be passive. For example, one or more of the colleagues can have a neutral one-way conversation with the user. For example, one or more of the colleagues can have a positive one-way conversation with the user. For example, one or more of the colleagues can have a negative one-way conversation with the user. For example, one or more of the colleagues can have conversation with the user asking questions about the burnout. For example, one or more of the colleagues can have a stressed attitude, e.g. having a stressed conversation about work overload with a third (virtual) party. For example, the colleagues can have a positive facial expression, a negative facial expression, or a neutral facial expression.

For example, the various parameters can include an attitude of a superior (avatar) in the virtual reality scenario. For example, the superior can be passive. For example, the superior can have a neutral, a positive, or a negative one-way conversation with the user or with a team (e.g. a briefing). For example, the superior can have a stressed attitude. For example, the superior can have a positive facial expression, a negative facial expression, or a neutral facial expression. For example, the superior can welcome the patient back in a way that is supportive, in a way that pressures the patient, or by giving the patient a new job and/or new responsibilities. For example, the superior may ask the patient about the burnout, e.g. what caused it and/or whether the patient will be able to function at 100% now.

For example, the various parameters can include a task parameter for the patient in the virtual reality scenario. A task parameter can relate to a task the patient is expected to perform in the virtual reality scenario. Optionally, the patient does not actually perform the task in the virtual reality scenario. The idea of being expected to perform the task, might be a trigger for the patient. The task can e.g. relate to reading an email and optionally replying to an email. The task can e.g. relate to opening an agenda. The task can e.g. relate to receiving a call, e.g. by telephone or digital (video) conferencing tools. The task can e.g. relate to, being invited to a meeting, or participating or organizing a meeting. The meeting can e.g. be online or in person. The task can e.g. relate to performing a presentation. The task parameter that the practitioner can select can e.g. relate to the type of task, and/or to the positivity of the task. The positivity of the task can e.g. be positive, neutral, or negative. The positivity can e.g. be affected by the attitude of other people and/or by the content of the task.

By selecting the various parameters, the practitioner can define various virtual reality scenarios, having different return-to-office scenarios. Optionally, a single return-to-office scenario has different scenes, wherein the different scenes optionally have different parameters.

In embodiments of the methods and/or systems, one of the return-to-office scenarios, e.g. the first return-to-office scenario, relates to the patient returning to work after an absence, wherein the patient is seated at a desk and faced with a plurality of unread emails and/or meeting requests. The patient may be e.g. at the office, e.g. in the presence of one or more colleagues or alone. The patient may e.g. be at home, e.g. a home-office. The practitioner may e.g. be able to select the location, the presence of colleagues, and the type of message (e.g. in the email title) being readable, e.g. by means of the parameters. Optionally, a first scene within the first return-to-office scenario relates to reading a first email, and a second scene relates to reading a second email.

In embodiments of the methods and/or systems, one of the return-to-office scenarios, e.g. the second return-to-office scenario, relates to the patient returning to the office after an absence, wherein the patient interacts with a plurality of a colleagues showing different levels of kindness. For example, one or more of the colleagues may welcome the patient back. For example, one or more of the colleagues may bring the patient up to date regarding events that happened during the absence. For example, one or more of the colleagues may ask how the patient is feeling in a friendly tone. For example, one or more of the colleagues may ask why the patient was absent in a critical tone. For example, one or more of the colleagues may make an unfriendly remark about the absence of the patient. The colleagues may e.g. be of the same as or lower level than the patient in the hierarchy of the company. The patient may e.g. interact with the colleagues at the office, e.g. at a desk or a relaxing location such as a watercooler, coffee machine, kitchen, or cafeteria. The patient may e.g. be at home, e.g. a home-office, interacting with the colleagues digitally. The practitioner can e.g. select the location, the number of colleagues, and/or the type of questions/conversation, e.g. with the parameters. Optionally, a first scene within the second return-to-office scenario relates to a conversation with a first colleague, and a second scene relates to a conversation with a second colleague.

In embodiments of the methods and/or systems, one of the return-to-office scenarios, e.g. the third return-to-office scenario, relates to the patient returning to the office after an absence, wherein the patient interacts with a superior. The superior may e.g. be a friendly superior. For example, the superior may welcome the patient back. The superior may e.g. be an unfriendly superior. For example, the superior may make an unfriendly comment, e.g. relating to the absence of the patient. The patient may e.g. interact with the superior at the office, e.g. at his desk or the desk of the superior. The patient may e.g. with the superior digitally, e.g. while being at a home-office. The practitioner can e.g. select the location, the number of conversations and/or the length of a conversation, and/or the kindness of the superior, e.g. with the parameters. Optionally, a first scene within the third return-to-office scenario relates to a conversation with a friendly superior, and a second scene relates to a conversation with an unfriendly superior (which may the same superior as the friendly superior or another superior).

In embodiments of the methods and/or systems, one of the return-to-office scenarios, e.g. the fourth return-to-office scenario relates to the patient returning to the office after an absence, and is required to participate and/or organise a meeting. The meeting may e.g. be an in-person meeting. The meeting may e.g. be an online meeting, e.g. using a videoconference tool. The practitioner can e.g. select the location, the type of meeting conversation, and/or the kindness of the superior, the number of attendees, the preparation time, e.g. with the parameters. Optionally, a first scene within the third return-to-office scenario relates to having to prepare a small meeting between colleagues, and a second scene relates to a larger meeting and/or a meeting with at least one superior.

In embodiments of the methods and/or systems, each virtual reality scenario includes a reflection element in which the patient sees a reflection of himself. For example, the virtual reality system may be configured to include the reflection element in the virtual reality scenario. The reflection element can e.g. be a mirror. The reflection element can e.g. be a window which shows a reflection. The reflection element may e.g. be shown in a starting phase of the virtual reality scenario. The reflection element may e.g. be present in the majority, optionally all, of the virtual reality scenario. The reflection may e.g. show an avatar. Optionally, the virtual reality system is configured to adapt the avatar based on the patient, e.g. to resemble the patient. The avatar may e.g. reflect a gender with which the patient identifies itself. The virtual reality system may e.g. be configured to adapt the avatar based on input provided by the patient and/or on an image of the patient (e.g. captured by the virtual reality system). Seeing a reflection of himself, may have the effect that the patient identifies better with the avatar. The virtual reality scenario may as such feel more “real” to the patient, and hence the burnout triggers as well.

In embodiments, the methods and/or systems may include or be configured to, presenting the first and second virtual reality scenario to the patient shortly after each other as a single virtual experience. Optionally, when present, also the third virtual reality scenario, and optionally the fourth virtual reality scenario, and optionally the fifth virtual reality scenario are presented as part of the same single virtual experience. Thus, the practitioner can select first which virtual reality scenarios will be presented to the patient, and then these will be presented as single virtual experiences. A single virtual experience can e.g. mean that no predetermined explicit pauses are present between two respective virtual reality scenarios. Optionally, a transition may be present between two respective virtual reality scenarios.

In embodiments the methods and/or systems may e.g. include or be configured to, (e.g. after presenting a virtual reality scenario to the patient and before starting a next scenario protocol; or while the virtual reality scenario is being paused): subjecting the patient to a plurality of questions related to his reaction to the burnout related triggers. The questions and the answers may e.g. be useful for the patient to assess how the patient reacts to the triggers, and why. The questions and the answers may e.g. be useful for the practitioner to discover and analyse how the patient reacts to the triggers, and why. Optionally, the practitioner decides based on this which virtual reality scenario and/or which scenario protocol is applied next.

In embodiments the methods and/or systems may e.g. include or be configured to, (e.g. after presenting a virtual reality scenario to the patient and before starting a next scenario protocol; or while the virtual reality scenario is being paused): subjecting the patient to an intellectual exercise for improving the reaction of the patient to the burnout related triggers. Improving the reaction can e.g. entail improving the cognition and/or giving the patient reassurance that the patient can do cognitive tasks while exposed to burnout triggers.

The intellectual exercise may e.g. be a Stroop-test. The intellectual exercise may e.g. be cognitive exercise, e.g. that should be relatively simple for the patient but still requires some mental concentration. Optionally, the intellectual exercise is not related to the work of the patient. Optionally, the intellectual exercise is not related to the burnout (or a relevant trigger) of the patient. For example, the intellectual exercise may be a mathematical problem at the level of a child (e.g. at elementary school), while the patient is an adult. It has been found that performing the intellectual exercise (which is relatively simple but yet forces the patient to think) shortly after exposing the patient to a trigger, helps to improve the reaction of the patient to triggers.

In embodiments the methods and/or systems, guidelines are provided to the practitioner. The guidelines are configured to assist the practitioner in applying the therapeutic process. The guidelines can e.g. provide information on when to apply which scenario protocol. The guidelines can e.g. provide information on how to apply a scenario protocol. The guidelines can e.g. be for the questions and/or intellectual exercise. The guidelines may help the practitioner in applying the questions and/or intellectual exercise. The guidelines may e.g. explain when to ask a question. The guidelines may e.g. explain which questions to ask and/or how the ask the questions. The guidelines may e.g. explain how to interpret the answers to the questions. The guidelines may e.g. explain how to apply the intellectual exercise. The guidelines may e.g. explain how to interpret the answers to the intellectual exercise.

In embodiments, the practitioner control system is configured to present at least a part of the guidelines to the practitioner, e.g. on the screen. For example, questions to be asked to the patient can be presented. This may facilitate the work of the practitioner.

In embodiments, at least one of the virtual reality scenarios is configured to require the patient to train a stress-reducing work-related competence. A stress-reducing work-related competence may e.g. include a soft skill. A stress-reducing work-related competence may e.g. include introducing himself, enumerating his own qualities, asking for help, saying no, greeting other people, accepting criticism, solve conflicts, show empathy. Optionally, a first virtual reality scenario is configured to train a stress-reducing work-related competence at a first training level, and a second virtual reality scenario is configured to train the stress-reducing work-related competence at a second training level, wherein the second training level is a higher training level than the first training level. Training the stress-reducing work-related competence may e.g. be achieved by putting the patient in a situation where he has (or should) apply the competence. A higher training level may entail that not applying the competence will cause more stress, or that is more difficult to apply the competence. For example, saying no to a superior may be more difficult than to a colleague at the same hierarchy level. Although it may be advantageous to combine these embodiments with the virtual reality scenarios having burnout relating triggers (at different levels), it is envisaged that these embodiments can also be applied as an alternative. That is, the invention can also relate to training stress-reducing work-related competences instead of (but also in addition to) subjecting the patient to burnout related triggers. These embodiments may be particularly advantageous when the patient was absent for stress-related reasons and/or common mental disorder instead of burnout, as explained in more detail further below. In such cases, the stress-reducing work-related competences may e.g. be linked to the specific mental disorder the patient suffers from.

Virtual reality scenarios wherein stress-reducing work-related competences are trained may be in similar settings as those mentioned herein for burn-put related triggers. For example, in a meeting with colleagues, a meeting with a boss, while working from home. However, it will be understood that it is possible that the virtual scenario can be a general work scenario, rather than being limited to a return-to-work scenario.

In embodiments the methods and/or systems may e.g. include or be configured to: visualising the practitioner as an avatar in the virtual reality scenario. The practitioner can thus participate in the virtual reality scenario. Optionally, the virtual reality system and the practitioner control system are configured to allow the practitioner to interact with the patient within the virtual reality scenario. For example, the practitioner can ask questions and/or perform an intellectual exercise within the virtual reality scenario.

In embodiments the methods and/or systems may e.g. include or be configured to: receiving audio input from the practitioner, and incorporating the audio input into the virtual reality scenario. For example, the practitioner can ask questions and/or perform an intellectual exercise within the virtual reality scenario.

In embodiments of the methods and/or systems, each virtual reality scenario is presented to the patient as a video clip, e.g. a 3D-video clip. The video clip is e.g. configured to be played using virtual reality glasses. The video clip is predetermined, meaning the events are independent of the actions of the patient. The video clip can e.g. be a 360-degree video clip.

In embodiments of the methods and/or systems, the patient is seated on a chair with wheels. The chair may e.g. be an office chair. Since the patient is seated at a desk or table at least at some point in most of the virtual reality scenarios, being seated on a chair with wheels can make the virtual experience feel more real. In addition, it has been found to make the calibration more position, the calibration relating to calibrating the position of the patient in real life to the position in the virtual scenario.

In embodiments the methods and/or systems may e.g. include or be configured to: calibrate the position of the patient in the virtual scenario. For example, the patient can be asked to stand up and sit again. The virtual reality system may include at least one positional sensor configured to detect a position of the virtual reality system (e.g. of the virtual reality glasses, the headset, the HMD). A control unit may e.g. be configured to detect a position of the patient when seated and when standing up (or a difference therebetween), and be configured to calibrate the position of the avatar of the patient accordingly. The calibration ensures that there is no mismatch between the different positions in the virtual reality scenario, which would otherwise reduce the effect of the experience.

In embodiments, the virtual reality system may include a control unit. The control unit can be configured to control various components of the virtual reality system, e.g. to perform various steps or actions. The control unit may e.g. control the virtual reality glasses, e.g. control what they show. The control unit may e.g. include one or more communication terminals for communication with other components. Such communication can e.g. occur via wired or wireless communication, using any of the suitable communication techniques or protocols. The control unit can e.g. be configured to communicate with a control unit of the practitioner control system and/or be configured to control the practitioner control system. The control unit can e.g. be configured to communicate with a central system for exchanging data and/or receiving updates. The central system can e.g. an online system and/or a cloud-based system. The control unit may e.g. include a memory for storing data, instructions, virtual reality scenarios, etc. The control unit may e.g. include a processing unit for performing processing steps.

In embodiments, the practitioner control system may include a control unit. The control unit can be configured to control various components of the practitioner system, e.g. to perform various steps or actions. The control unit may e.g. include one or more communication terminals for communication with other components. Such communication can e.g. occur via wired or wireless communication, using any of the suitable communication techniques or protocols. The control unit can e.g. be configured to communicate with a control unit of the virtual reality system and/or be configured to control the virtual reality system. The control unit can e.g. be configured to communicate with a central system for exchanging data and/or receiving updates. The central system can e.g. an online system and/or a cloud-based system. The control unit may e.g. include a memory for storing data, instructions, virtual reality scenarios, etc. The control unit may e.g. include a processing unit for performing processing steps.

In embodiments, the virtual reality system includes one or two handles, wherein a handle is configured to be held by the patient in a hand. The handle is configured to allow the patient to point to an object in the virtual environment. The virtual reality system is e.g. configured to allow the patient to select items using the handle. For example, the patient can select an option and/or can select an item on a screen within a virtual reality scenario. In embodiments, the method is performed multiple times on different days for treating the patient. In embodiments, the system is configured to present the first and/or second virtual reality scenario to the patient multiple times on different days. The treatment of the patient is thus not performed in a single session, but by multiple sessions on different days. The treatment is part of a therapeutic process. The practitioner can evaluate the progress of the patient and adapt the levels of the burnout triggers accordingly (by selecting the appropriate virtual reality scenario). When the patient has improved sufficiently, the practitioner can decide the patient is ready to actually return to work.

In embodiments, a virtual reality scenario can last between 20 second and 5 minutes.

Various embodiments have been elaborated on above. It will be understood that these embodiments can be combined with each other to achieve additional advantages, even is their combination has not been explicitly made in this text or in the appended claims.

Although the description above was aimed at patients suffering from burnout returning to work, it is envisaged that principles of the system can be applied generally to person returning to work after an absence. The absence can e.g. be stress-related, and/or e.g. be related to a common mental disorder. In such cases, any references made above to burnout related triggers can be considered as meaning absence related triggers. Such absence related trigger can e.g. be a stress trigger, which e.g. is of a type of trigger that contributed to a stress-related absence. In case of a common mental disorder related absence, the absence related trigger may be dependent on the type of mental disorder. Below, several general embodiments relating to this principle are explicitly incorporated. It will be understood, however, that any of the embodiments or features thereof as described above, may also be applied to the embodiments below, mutatis mutandis.

In embodiments, the invention relates to a method for preparing a person to return to work after an absence, e.g. a stress-related absence or an absence related to a common mental disorder, the method including:
applying a first scenario protocol, including:
controlling, using a practitioner control system, when a virtual reality system including virtual reality glasses presents a first virtual reality scenario to a patient, wherein:
the first virtual reality scenario represents a first return-to-office scenario, wherein the first return-to-office scenario includes one or more absence related triggers at a first level;
presenting the first virtual reality scenario to the patient using the virtual reality glasses arranged on the patient, wherein the patient virtually experiences the first virtual reality scenario in first person;
while the first virtual reality scenario is being presented to the patient by the virtual reality system, visually presenting the first virtual reality scenario to a practitioner using a screen;
applying a second scenario protocol, including:
controlling, using the practitioner control system, when the virtual reality system including virtual reality glasses presents a second virtual reality scenario to the patient, wherein:
the second virtual reality scenario represents a second return-to-office scenario, wherein the second return-to-office scenario includes one or more absence related triggers at a second level;
presenting the second virtual reality scenario to the patient using the virtual reality glasses arranged on the patient, wherein the patient virtually experiences the second virtual reality scenario in first person;
while the second virtual reality scenario is being presented to the patient by the virtual reality system, visually presenting the second virtual reality scenario to the practitioner using the screen;
wherein
the triggers at the second level in the second return-to-office scenario are at a higher stress level in comparison to the triggers at the first level in the first return-to-office scenario.

In embodiments, the invention relates to a method for preparing a person to return to work after an absence, e.g. a stress-related absence or an absence related to a common mental disorder, the method including:
controlling a practitioner control system to:
allow a practitioner to control a virtual reality system including virtual reality glasses when to present a first virtual reality scenario and when to present a second virtual reality scenario;
controlling a virtual reality system to, in function of the control provided by the practitioner control system:
present the first virtual reality scenario to the patient using the virtual reality glasses arranged on the patient, wherein
the patient virtually experiences the first virtual reality scenario in first person;
the first virtual reality scenario represents a first return-to-office scenario, wherein the first return-to-office scenario includes one or more absence related triggers at a first level;
present the second virtual reality scenario to the patient using the virtual reality glasses arranged on the patient, wherein
the patient virtually experiences the second virtual reality scenario in first person;
the second virtual reality scenario represents a second return-to-office scenario, wherein the second return-to-office scenario includes one or more absence related triggers at a second level;
control the practitioner control system to:
while the first virtual reality scenario is being presented to the patient by the virtual reality system, visually present the first virtual reality scenario to a practitioner using a screen;
while the second virtual reality scenario is being presented to the patient by the virtual reality system, visually present the second virtual reality scenario to the practitioner using the screen;
wherein
the triggers at the second level in the second return-to-office scenario are at a higher stress level in comparison to the triggers at the first level in the first return-to-office scenario.

In embodiments, the invention relates to a system for preparing a person to return to work after an absence, e.g. a stress-related absence or an absence related to a common mental disorder, the system including:
a practitioner control system configured to:
allow a practitioner to control a virtual reality system including virtual reality glasses when to present a first virtual reality scenario and when to present a second virtual reality scenario;
a virtual reality system configured to, in function of the control provided by the practitioner control system:
present the first virtual reality scenario to the patient using the virtual reality glasses arranged on the patient, wherein:
the patient virtually experiences the first virtual reality scenario in first person;
the first virtual reality scenario represents a first return-to-office scenario, wherein the first return-to-office scenario includes one or more stress related triggers at a first level;
present the second virtual reality scenario to the patient using the virtual reality glasses arranged on the patient,
the patient virtually experiences the second virtual reality scenario in first person;
the second virtual reality scenario represents a second return-to-office scenario, wherein the second return-to-office scenario includes one or more stress related triggers at a second level
wherein the practitioner control system is further configured to:
while the first virtual reality scenario is being presented to the patient by the virtual reality system, visually present the first virtual reality scenario to a practitioner using a screen;
while the second virtual reality scenario is being presented to the patient by the virtual reality system, visually present the second virtual reality scenario to the practitioner using the screen;
wherein
the triggers at the second level in the second return-to-office scenario are at a higher stress level in comparison to the triggers at the first level in the first return-to-office scenario.

In embodiments, the invention relates to non-transitory computer readable instructions, configured to cause a system according any of the embodiments described herein to perform the method according to any of the embodiments described herein.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1: schematically illustrates a system for preparing a patient suffering from burnout to return to work with a therapeutic process by applying exposure therapy;

FIG. 2: schematically illustrates a method for preparing a patient suffering from burnout to return to work with a therapeutic process by applying exposure therapy.

### DETAILED DESCRIPTION OF THE INVENTION

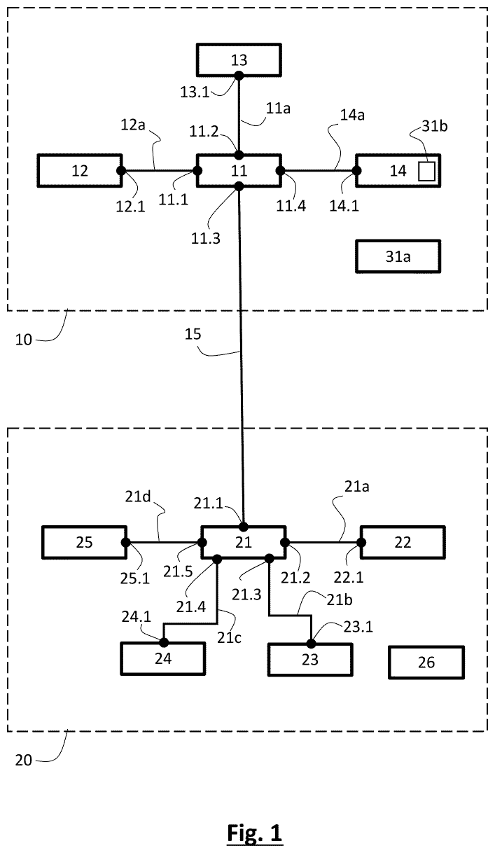

FIG. 1 schematically illustrates a system for preparing a patient suffering from burnout to return to work with a therapeutic process by applying exposure therapy. A practitioner control system 10 and a virtual reality system 20 are illustrated. The practitioner control system 10 includes a practitioner control unit 11, which can be embodied in any suitable manner. The practitioner control system 10 further includes a screen 13 and an input module 12.

The practitioner control system 10 allows a practitioner, e.g. a therapist or psychologist, to interact. The practitioner can e.g. provide inputs via input module 12, which can e.g. be a keyboard and/or mouse and/or touch screen. The inputs provided by the practitioner are transmitted to the practitioner control unit 11 via input signal 12a, which is transmitted via communication terminal 12.1 and 11.1.

Information can be provided to the practitioner via the screen 13. The screen can e.g. be a screen of a computer, tablet, or smartphone. The practitioner control unit 11 generates screen signal 11a, which is transmitted to the screen via communication terminals 11.2 and 13.1.

In embodiments, the input module 12 and the screen 13 can be combined in a touch screen. In embodiments, the input module 12, the screen 13, and the practitioner control unit 11 can be combined in a single system, e.g. a computer system or a tablet system.

The virtual reality system 20 includes a VR control unit 21, which can be embodied in any suitable manner. The virtual reality system 20 includes virtual reality glasses 22. The virtual reality glasses 22 can e.g. be integrated in a headset or HMD, which may further include a sound emitter 24 for emitting sounds. The virtual reality glasses 22 (e.g. the headset) can be worn by a patient of the practitioner. The virtual reality system 20 further includes at least one handle 25, which the patient can hold in a hand and use to point to something or select something in the virtual environment. Although the handle 25 will further be discussed in singular form, it will be understood that the virtual reality system 20 may include a first handle 25 for the left hand of the patient and a second handle 25 for the second hand of the patient.

The VR control unit 21 can control what is presented to the patient, by controlling the virtual reality glasses 22 with control signal 21a. Control signal 21a is transmitted via communication terminals 21.2 and 22.1. The control signal 21a can e.g. include instructions as to which virtual reality scenario to present to the patient, when to start playing, when to stop playing, and when to pause the virtual reality scenario. Similarly, the VR control unit 21 can control what is emitted by the sound emitter 24 by means of control signal 21c, which is transmitted via communication terminals 21.4 and 24.1. Communication signal 21d (transmitted via communication terminals 25.1 and 21.5 provides the VR control unit 21 with input the user gives via the handle 25. The input can e.g. be movement of the handle 25 and or activating a button on the handle 25. The handle 25 can e.g. include a positional sensor and/or a gyroscope sensor to determine the movement.

In the shown embodiment, the virtual reality system 20 includes a memory 23, on which a plurality of virtual reality scenarios are stored. The virtual reality scenarios may e.g. 3D video clips, e.g. 360-degree video clips. The VR control unit 21 can communicate with the memory via communication signal 21b, which is transmitted via communication terminals 21.3 and 23.1. The VR control unit 21 can e.g. request the memory 23 to provide a virtual reality scenario, which is then transmitted by the VR control unit 21 to the virtual reality glasses via control signal 21a and to the sound emitter 24 via control signal 21c. In other embodiments, it is possible that a direct communicative connection is established between the memory 23 and the virtual reality glasses 22 and the sound emitter 24 for transmitting the selected virtual reality scenario.

The practitioner control system 10 and the virtual reality system 20 can communicate with each other via communication signal 15. The communication signal 15 is in particular transmitted between the practitioner control unit 11 and the VR control unit 21 via communication terminals 11.3 and 21.1. This allows the practitioner to control the virtual reality glasses 22, by providing an input on input module 12 which communicated (optionally after suitable conversion/processing) via the practitioner control unit 11 and communication signal 15 to the VR control unit 21. The VR control unit 21 then generates the control signals 21a, 21b, 21c based thereon. The input that the practitioner provides on the input module 12 can e.g. relate to which virtual reality scenario to be played. The practitioner can e.g. finetune the virtual reality scenario by setting various parameters of the virtual reality scenario.

The communication signal 15 is also used to communicate data from the virtual reality system 20 to the practitioner control system 10. In particular, the virtual reality scenario that is presented to the patient via virtual reality glasses 22, is also transmitted to the practitioner control system 10. The virtual reality scenario is presented to the practitioner on the screen 13, while it is being presented to the patient. This allows the practitioner to follow along with the virtual reality scenario, and to interpret the reactions of the patient. The practitioner can also pause the virtual reality scenario when considering that appropriate, by giving a pause command on the input module 12.

The various signals (e.g. 11a, 12a, 14a, 15, 21a, 21b, 21c, 21d) can be transmitted using any of the suitable communication techniques, including wired and wireless communication techniques. In particular for the communication signal 15 it can be advantageous to use a wireless communication technique, e.g. Wi-Fi or Bluetooth, although a cable for a wired communication can also be provided.

The practitioner control system 10 also includes a memory 14. Information can be communicated between the memory 14 and the practitioner control unit 11 via communication signal 14a, which is transmitted via communication terminals 11.4 and 14.1. The memory 14 can include various stored information, including a manual 31b. The manual 31b is digital and can be depicted on the screen 13, in addition or alternatively to the digital manual 31b, the practitioner control system 10 can also include a hardcopy manual 31a on printed on paper.

The manuals 31a, 31b can include guidelines for the practitioner. Such guidelines can explain how to use the virtual reality system 20, e.g. when to present which virtual reality scenario to the patient. The guidelines can also include questions that the practitioner can ask the patient, e.g. during or after having seen a virtual reality scenario. The guidelines can also include instructions for performing intellectual exercises with the patient.

FIG. 1 further schematically illustrates that the virtual reality system 20 may include a chair 26. The chair 26 is an office chair with wheels. The patient will be seated on the chair while using the virtual reality system 20.

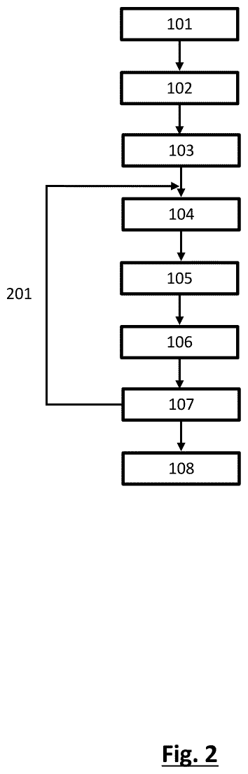

FIG. 2 schematically illustrates a method for preparing a patient suffering from burnout to return to work with a therapeutic process by applying exposure therapy. The method in FIG. 2 can be performed with the system shown in FIG. 1, although neither is limited thereto. In the following explanation, the process will be explained step by step by way of example. It will be understood, however, that this should not be construed as limiting to the scope of the invention. The invention does not require each step to be present in all embodiments. Moreover, a method performed by the practitioner may include some of explained steps, while other steps may be performed by other actors. Similarly, a method performed by the system may include some of the explained steps, although maybe formulated slightly different, mutatis mutandis.

In step 101, a patient is diagnosed with a burnout. The patient is in particular a person who works in an office environment, e.g. at a desk job. Although the causes leading to a burnout are complex and usually come from different sources, stress related to the work/office environment is often a contributing factor. The burnout can thus an occupational burnout. The burnout will usually cause the patient to stay home from work for a prolonged time.

In step 102, the patient visits a practitioner for establishing a therapeutic process. The therapeutic process may be part of a care pathway that is agreed upon between the practitioner and the patient. The practitioner can e.g. be a therapist or psychologist. The practitioner can e.g. be somebody who is trained to use a system as e.g. shown in FIG. 1. The practitioner may have a first introductory conversation with the patient to discuss the current state of the burnout, triggers that have contributed to the burnout, and how returning to the office in the future may (re-)trigger the burnout. The practitioner may determine a therapeutic process, e.g. (at least partially) based on the conversation with the patient. The therapeutic process includes showing a plurality of virtual reality scenarios to the patient, wherein each virtual reality scenario represents a return-to-office scenario having one or more burnout related triggers. The practitioner may optionally determine the therapeutic process using a manual (e.g. manual 31a or 31b shown in FIG. 1). The practitioner may determine various aspects of the virtual reality scenario by setting various parameters.

In step 103 the virtual reality system 20 is set up for the patient and calibrated. It is possible that the setting up and calibrating is done during the same visit as explained with reference to step 102, or in a later visit.

Setting up and calibrating may e.g. include the practitioner providing detail of the patient into the input module 12. The details may e.g. include the name of the patient. Setting up and calibrating may e.g. include an introduction to using the handle 25. For example, the virtual reality glasses 22 may show a few objects on different locations, which the patient has to select using the handle 25.

Setting up and calibrating may e.g. include selecting a preferred gender of the patient and/or a selecting a preferred language of the patient. This can e.g. be done by the practitioner by inputting this in the input module 12, but it is also possible that the patient can select this within the virtual reality system 20, e.g. using the handle 25.

Setting up and calibrating may e.g. include calibrating a body position of the patient. The body position of the patient in the virtual reality environment may be calibrated to the body position in real life. This may make the virtual experience feel more real. It may also be helpful to make sure that a change of body position in real life will be represented by a similar change in position in the virtual environment. The calibration can e.g. be done by asking the patient to sit (e.g. on a chair with wheels) and stand up. The virtual reality system 20 may e.g. include one or more sensors (e.g. positional sensors and/or gyroscope sensors), which may e.g. be arranged in the headset of HMID 22. The movement by the patient can be sensed with the sensors and transmitted to the VR control unit 21 via signal 21a. The VR control unit 21 can calibrate the position of the patient in the virtual environment based on the signal of the sensor.

In step 104, the practitioner applies a first scenario protocol. The first scenario protocol includes presenting a first virtual reality scenario to the patient. The practitioner can control, e.g. with the practitioner control system 10, when the first virtual reality scenario is presented to the patient via the virtual reality system 20. While the first virtual reality scenario is presented to the patient in first person and in virtual reality, the practitioner can follow along on the screen 13. Optionally, the patient is seated on a chair 26 with wheels while being subjected to the first virtual reality scenario.

The first virtual reality scenario includes burnout related triggers at a first level. The first return-to-office scenario can e.g. relate to the patient returning to work after an absence, wherein the patient is seated at a desk and faced with a plurality of unread emails and meeting requests. A screen (e.g. computer screen or tablet screen) may e.g. be arranged on the desk, showing a mailbox and a calendar. The practitioner can select various parameters of the first virtual reality scenario. These parameters can e.g. relate to the environment. The patient may e.g. at the office, e.g. in the presence of one or more colleagues in an open office or alone. The patient may e.g. be at home, e.g. a home-office, e.g. at a kitchen or dining table with a laptop arranged thereon. The various parameters can also include task parameters. The task parameters can e.g. relate to the emails and meeting request, e.g. determining the topic, sending party, and the time pressure. The first return-to-office scenario may include multiple scenes, with different (task) parameters. For example, the unread emails may be from different parties (e.g. boss, colleague, client) and relate different issues (e.g. neutral or negative issues). This can be set by the practitioner with the parameters via the practitioner control system 10, and then the patient can select an email accordingly. For example, the meeting requests may be different types of meetings, e.g. online or in-person meetings, e.g. a meeting with one colleague, a meeting with a small team, a meeting with a large group of the company, an individual meeting with the boss. There may also be a timing conflict between two meetings. The practitioner can select the parameters accordingly. In a first scene, everything may be more positive, and in subsequent scenes, more stressful events may occur.

The patient is thus faced with a return-to-office scenario, which due to being presented in virtual reality feels quite real to the patient. However, the patient is in a controlled environment, and the virtual reality scenario is limited in time and can be paused at any moment. This allows the patient to better understand his reaction to the triggers. Together with the practitioner, the patient can find ways to improve the reaction to the triggers. In particular, the reaction can be re-programmed. As such, the patient is subjected to exposure therapy.

During the first virtual reality scenario, a reflective element may be provided which shows a reflection of the patient, e.g. of an avatar of the patient. This has been found to improve the perception of the patient regarding the virtual reality scenario. Optionally the avatar resembles the patient, e.g. at least the gender. The avatar can e.g. be based on input provided by the patient, wherein the patient selected the avatar and/or body parts of the avatar. It is also possible that the system automatically generates the avatar based on an image of the patient. The reflective element may e.g. be a window in the virtual environment (e.g. at the open office, in the home office, or in a meeting room). This is a practical implementation of the reflective element, since a mirror might seem unnatural in the environments in which the virtual reality scenarios occur.

The first scenario protocol may further include that the practitioner asks several questions to the patient. The practitioner can e.g. ask these questions after the virtual reality scenario has finished, but it may also be possible that the practitioner pauses the virtual reality scenarios to ask the questions. The questions may help the patient and/or the practitioner to better understand the reaction of the patient to the triggers. The questions can also help the patient to improve his reaction to the triggers. In some embodiments, the questions (or guidelines regarding the questions) can be presented to the practitioner on the screen.

The first scenario protocol may further include that the practitioner performs an intellectual exercise with the patient. The intellectual exercise can e.g. be performed after the virtual reality scenario has finished, but it may also be possible that the practitioner pauses the virtual reality scenarios. The intellectual exercise may e.g. be a Stroop-test. The intellectual exercise may e.g. be cognitive exercise, e.g. that should be relatively simple for the patient but still requires some mental concentration. Optionally, the intellectual exercise is not related to the work of the patient. Optionally, the intellectual exercise is not related to the burnout (or a relevant trigger) of the patient.

In some embodiments, the practitioner may ask the questions and/or perform the intellectual exercise in a normal (face to face) conversational way with the patient. The patient may e.g. remove the virtual reality glasses 22 to do so. In other embodiments, it is possible that the practitioner is represented by an avatar in the virtual reality scenario, such that the interaction between practitioner and patient can be done in the virtual reality environment. In some embodiments, the practitioner provides audio input into the practitioner control system 10, which may be communicated to the virtual reality system 20 and incorporated into the virtual reality scenario via sound emitter 24.

After having concluded the first scenario protocol, the practitioner may apply a second scenario protocol in step 105. The second scenario protocol may in many ways be similar to the first scenario protocol, but a second virtual reality scenario is presented to the patient instead of the first virtual reality scenario. It will be understood that different questions and/or intellectual exercise(s) may be suitable for the second virtual reality scenario compared to the first scenario protocol.

The second virtual scenario is also a return-to-office scenario, but in this case the burnout related triggers are at a second level. The second level may be a higher stress level than the first level. For example, the second return-to-office scenario may relate to the patient returning to the office after an absence, wherein the patient interacts with a plurality of a colleagues showing different levels of kindness. For example, one or more of the colleagues may welcome the patient back. For example, one or more of the colleagues may bring the patient up to date regarding events that happened during the absence. For example, one or more of the colleagues may ask how the patient is feeling in a friendly tone. For example, one or more of the colleagues may ask why the patient was absent in a critical tone. For example, one or more of the colleagues may make an unfriendly remark about the absence of the patient. The colleagues may e.g. be of the same as or lower level than the patient in the hierarchy of the company. The practitioner can e.g. select the kindness of the colleagues via parameters inputted into the input module 12. Different conversation with different colleagues can be part of the second return-to-office scenario as different scenes. The practitioner can also select the environment via the parameters. The patient may e.g. interact with the colleagues at the office, e.g. at a desk or a relaxing location such as a watercooler, coffee machine, kitchen, or cafeteria. The patient may e.g. be at home, e.g. a home-office, interacting with the colleagues digitally.

In step 106, the practitioner may apply a third scenario protocol, including the presenting of a third virtual reality scenario which is again a return-to-office scenario. The third return-to-office scenario may e.g. relate to the patient returning to the office after an absence, wherein the patient interacts with a superior. The practitioner can e.g. select the level of kindness of the superior via the parameters. For example, the superior may welcome the patient back. For example, the superior may make an unfriendly comment, e.g. relating to the absence of the patient. For example, the superior may assign the patient to a new function or new responsibilities. The virtual reality scenario may e.g. include different scenes wherein a superior has different level of kindness. This may e.g. be the same superior asking more questions, or another superior. The practitioner can also select the environment via the parameters. The patient may e.g. interact with the superior at the office, e.g. at his desk or the desk of the superior. The patient may e.g. with the superior digitally, e.g. while being at a home-office.

In step 107, the practitioner may apply a fourth scenario protocol, including the presenting of a fourth virtual reality scenario which is again a return-to-office scenario. The fourth return-to-office scenario may e.g. relate to the patient returning to the office after an absence, and is required to participate and/or organise a meeting. The practitioner can e.g. select the environment of the meeting and the participants with the parameters. The meeting may e.g. be an in-person meeting, e.g. in a meeting room. The meeting may e.g. be an online meeting, e.g. using a videoconference tool. When the meeting is done in a meeting room, the meeting room may e.g. include one or more motivational posters that have stress-triggering content. The content may e.g. relate to working hard, profitability, performance, etc. The meeting may e.g. be a meeting with colleagues, a meeting with higher level management, a meeting with a client, a meeting with a prospect.

Although in this example four scenario protocols are applied, it will be understood that more or less may also be possible. In the shown example, the five scenario protocols are applied at the same day, e.g. during the same visit of the patient at the practitioner, but it may also be possible to spread this over multiple visits and/or days.

It can further be seen that, in this example, the levels of the burnout related triggers in each subsequent scenario protocol are at a higher level than at the preceding scenario protocol. This may be an advantageous therapeutic process to prepare the patient to return to the office. However, it is also possible to provide a different order. For example, after applying a virtual reality scenario with triggers at a high level, a virtual reality scenario with triggers at a low level is presented to the patient.

Usually the therapeutic process includes that the patient is exposed to at least some of the scenario protocols more than once. As illustrated by arrow 201 in FIG. 2, the method may restart from step 104 and again apply the four scenario protocols (although it may also be possible to apply less and/or slightly different scenario protocols, e.g. the practitioner may adapt the parameters for this). This can be done on a different day during a different visit.

If the practitioner decides that the patient is ready, the patient may return to the office in step 108. The office where the patient returns to can be the same office as where he worked previously, but it can also be another office.

Although the figures and associated description above was aimed at patients suffering from burnout returning to work, it is envisaged that principles of the system can be applied generally to person returning to work after an absence. The absence can e.g. be stress-related, and/or e.g. be related to a common mental disorder. In such cases, any references made above to burnout related triggers can be considered as meaning absence related triggers. Such absence related trigger can e.g. be a stress trigger, which e.g. is of a type of trigger that contributed to a stress-related absence. In case of a common mental disorder related absence, the absence related trigger may be dependent on the type of mental disorder.

As required, detailed embodiments of the present invention are described herein; however, it is to be understood that the disclosed embodiments are merely examples of the invention, which may be embodied in various ways. Therefore, specific structural and functional details disclosed herein are not to be construed as limiting, but merely as a basis for the claims and as a representative basis for teaching those skilled in the art to practice the present invention in various ways in virtually any suitable detailed structure. Not all of the objectives described need be achieved with particular embodiments.

Furthermore, the terms and expressions used herein are not intended to limit the invention, but to provide an understandable description of the invention. The words “a”, “an”, or “one” used herein mean one or more than one, unless otherwise indicated. The terms “a multiple of”, “a plurality” or “several” mean two or more than two. The words “include”, “include”, “contain” and “have” have an open meaning and do not exclude the presence of additional elements. Reference numerals in the claims should not be construed as limiting the invention.

The mere fact that certain technical features are described in different dependent claims still allows the possibility that a combination of these technical measures can be used advantageously.

A single processor or other unit can perform the functions of various components mentioned in the description and claims, e.g. of processing units or control units, or the functionality of a single processing unit or control unit described herein can in practice be distributed over multiple components, optionally physically separated of each other. Any communication between components can be wired or wireless by known methods.

The actions performed by the control unit can be implemented as a program, for example computer program, software application, or the like. The program can be executed using computer readable instructions. The program may include a subroutine, a function, a procedure, an object method, an object implementation, an executable application, a source code, an object code, a shared library/dynamic load library and/or other set of instructions designed for execution on a computer system.

A computer program or computer-readable instructions can be stored and/or distributed on a suitable medium, such as an optical storage medium or a solid-state medium supplied with or as part of other hardware, but can also be distributed in other forms, such as via internet or other wired or wireless telecommunication systems.

Of note, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes”, and/or “including,” when used in this specification, specify the presence of stated features, integers, steps, operations, elements, and/or components, but do not preclude the presence or addition of one or more other features, integers, steps, operations, elements, components, and/or groups thereof.

As well, the corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

1. A method for preparing a patient suffering from burnout to return to work with a therapeutic process by applying exposure therapy, comprising:
applying a first scenario protocol, comprising:
controlling, using a practitioner control system, when a virtual reality system comprising virtual reality glasses presents a first virtual reality scenario to a patient, wherein:
the first virtual reality scenario represents a first return-to-office scenario, wherein the first return-to-office scenario comprises one or more burnout related triggers at a first level;
presenting said first virtual reality scenario to the patient using the virtual reality glasses arranged on the patient, wherein the patient virtually experiences the first virtual reality scenario in first person;
while the first virtual reality scenario is being presented to the patient by the virtual reality system, visually presenting the first virtual reality scenario to a practitioner using a screen;
applying a second scenario protocol, comprising:
controlling, using the practitioner control system, when the virtual reality system comprising virtual reality glasses presents a second virtual reality scenario to the patient, wherein:
the second virtual reality scenario represents a second return-to-office scenario, wherein the second return-to-office scenario comprises one or more burnout related triggers at a second level;
presenting said second virtual reality scenario to the patient using the virtual reality glasses arranged on the patient, wherein the patient virtually experiences the second virtual reality scenario in first person;
while the second virtual reality scenario is being presented to the patient by the virtual reality system, visually presenting the second virtual reality scenario to the practitioner using the screen;
wherein
the burnout related triggers at the second level in the second return-to-office scenario are at a higher stress level in comparison to the burnout related triggers at the first level in the first return-to-office scenario.

2. The method according to claim 1, further comprising
applying a third scenario protocol, comprising the presenting of a third virtual reality scenario representing a third return-to-office scenario comprising one or more burnout related triggers at a third level, wherein said third level is at a higher stress level in comparison to the burnout related triggers in the second return-to-office scenario;
applying a fourth scenario protocol, comprising the presenting of a fourth virtual reality scenario representing a fourth return-to-office scenario comprising one or more burnout related triggers at a fourth level, wherein said fourth level is at a higher stress level in comparison to the burnout related triggers in the third return-to-office scenario.

3. The method according to claim 1, wherein in each virtual reality scenario comprises a reflective surface in which the patient sees a reflection of himself.

4. The method according to claim 1, wherein the practitioner can select multiple parameters for each virtual reality scenario, including one or more of: an environment in which the virtual reality scenario occurs, an attitude of one or more colleagues in the virtual reality scenario; an attitude of a superior in the virtual reality scenario; a task parameter for the patient in the virtual reality scenario.

5. The method according to claim 1, wherein each virtual reality scenario comprises a plurality of scenes, wherein at least one of the parameters is different between subsequent scenes.

6. The method according to claim 1, wherein the first scenario protocol is applied at a point in time before the second scenario protocol is applied, and optionally:
the third scenario protocol is applied at a point in time after the second scenario protocol is applied; and/or
the fourth scenario protocol is applied at a point in time after the third scenario protocol is applied; and/or
the fifth scenario protocol is applied at a point in time after the fourth scenario protocol is applied.

7. The method according to claim 1, wherein the first and second virtual reality scenario are presented to the patient shortly after each other as a single virtual experience.

8. The method according to claim 1, wherein
the first return-to-office scenario relates the patient returning to the office after an absence, wherein the patient is seated at a desk and faced with a plurality of unread emails and/or meeting requests;
the second return-to-office scenario relates to the patient returning to the office after an absence, wherein the patient interacts with a plurality of a colleagues showing different levels of kindness.

9. The method according to claim 2, wherein
the third return-to-office scenario relates to the patient returning to the office after an absence, wherein the patient interacts with a superior;
the fourth return-to-office scenario relates to the patient returning to the office after an absence, and is required to participate and/or organise a meeting.

10. The method according to claim 1, wherein at least one of the virtual reality scenarios is configured to require the patient to train a stress-reducing work-related competence, e.g. introducing himself, enumerating own qualities, asking for help, saying no, greeting other people, accepting criticism, solve conflicts, show empathy.

11. The method according to claim 1, wherein each virtual reality scenario comprises:
subjecting the patient to a plurality of questions related to his reaction to the burnout related triggers, and/or
subjecting the patient to an intellectual exercise for improving the reaction of the patient to the burnout related triggers.

12. The method according to claim 9, wherein guidelines for the questions and/or intellectual exercise are provided to the practitioner.

13. The method according to claim 1, further comprising: visualising the practitioner as an avatar in the virtual reality scenario.

14. The method according to claim 1, further comprising: receiving audio input from the practitioner, and incorporating the audio input into the virtual reality scenario.

15. The method according to claim 1, wherein each virtual reality scenario is presented to the patient as a video clip, e.g. a 3D-video clip and/or a 360 degrees video clip.

16. The method according to claim 1, wherein the patient is seated on a chair with wheels.

17. The method according to claim 1, wherein the method is performed multiple times on different days for treating the patient.

18. A method for preparing a patient suffering from burnout to return to work with a therapeutic process by applying exposure therapy, comprising:
controlling a practitioner control system to:
allow a practitioner to control a virtual reality system comprising virtual reality glasses when to present a first virtual reality scenario and when to present a second virtual reality scenario;
controlling a virtual reality system to, in function of the control provided by the practitioner control system:
present said first virtual reality scenario to the patient using the virtual reality glasses arranged on the patient, wherein
the patient virtually experiences the first virtual reality scenario in first person;
the first virtual reality scenario represents a first return-to-office scenario, wherein the first return-to-office scenario comprises one or more burnout related triggers at a first level;
present said second virtual reality scenario to the patient using the virtual reality glasses arranged on the patient, wherein
the patient virtually experiences the second virtual reality scenario in first person;
the second virtual reality scenario represents a second return-to-office scenario, wherein the second return-to-office scenario comprises one or more burnout related triggers at a second level;
control the practitioner control system to:
while the first virtual reality scenario is being presented to the patient by the virtual reality system, visually present the first virtual reality scenario to a practitioner using a screen;
while the second virtual reality scenario is being presented to the patient by the virtual reality system, visually present the second virtual reality scenario to the practitioner using the screen;
wherein
the burnout related triggers at the second level in the second return-to-office scenario are at a higher stress level in comparison to the burnout related triggers at the first level in the first return-to-office scenario.

19. A method for preparing a person to return to work after an absence, e.g. a stress-related absence or an absence related to a common mental disorder, the method comprising:
controlling a practitioner control system to:
allow a practitioner to control a virtual reality system comprising virtual reality glasses when to present a first virtual reality scenario and when to present a second virtual reality scenario;
controlling a virtual reality system to, in function of the control provided by the practitioner control system:
present said first virtual reality scenario to the patient using the virtual reality glasses arranged on the patient, wherein
the patient virtually experiences the first virtual reality scenario in first person;
the first virtual reality scenario represents a first return-to-office scenario, wherein the first return-to-office scenario comprises one or more absence related triggers at a first level;
present said second virtual reality scenario to the patient using the virtual reality glasses arranged on the patient, wherein
the patient virtually experiences the second virtual reality scenario in first person;
the second virtual reality scenario represents a second return-to-office scenario, wherein the second return-to-office scenario comprises one or more absence related triggers at a second level;
control the practitioner control system to:
while the first virtual reality scenario is being presented to the patient by the virtual reality system, visually present the first virtual reality scenario to a practitioner using a screen;
while the second virtual reality scenario is being presented to the patient by the virtual reality system, visually present the second virtual reality scenario to the practitioner using the screen;
wherein
the triggers at the second level in the second return-to-office scenario are at a higher stress level in comparison to the triggers at the first level in the first return-to-office scenario.
